(19) United States
(12) Patent Application Publication
(10) Pub. No.: US 2012/0197185 A1
(43) Pub. Date: Aug. 2, 2012

(54) Electromechanical system for IV control

(76) Inventor: Kai Tao, Yizheng (CN)

(21) Appl. No.: 13/019,698

(22) Filed: Feb. 2, 2011

(51) Int. Cl. A61M 5/168

(52) U.S. Cl. 604/67

(57) ABSTRACT

We describe electromechanical systems for IV control. Based on dripping speed measurement by video processing technique, we adjust the position of the presser to press the dripping tube to the appropriate position to control the dripping speed. To do this mechanically, the processing unit issues commands to a stepper motor to control its rotation, and either a leadscrew or its variants, or a cam, is used to translate motor's rotation into presser's linear motion. When the processing unit detects that the dripping is finished, the device will also cut the dripping off immediately with the presser.

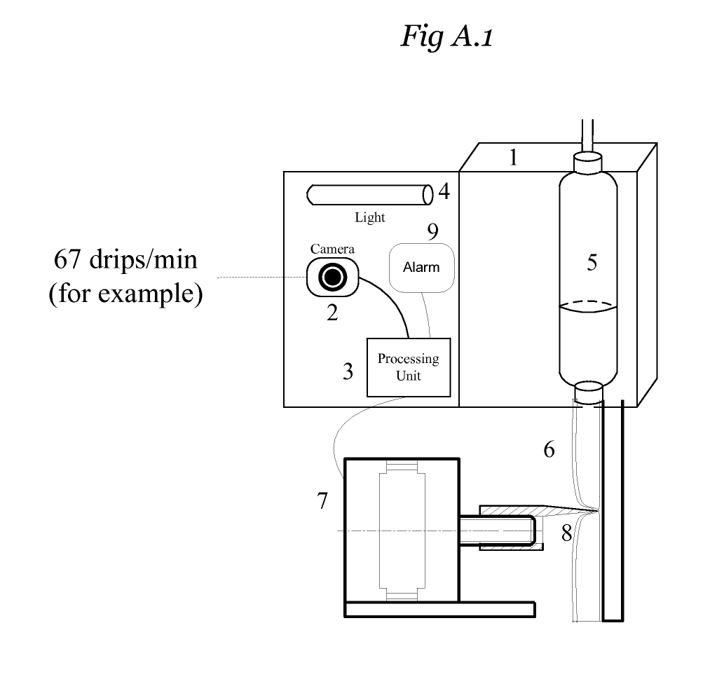

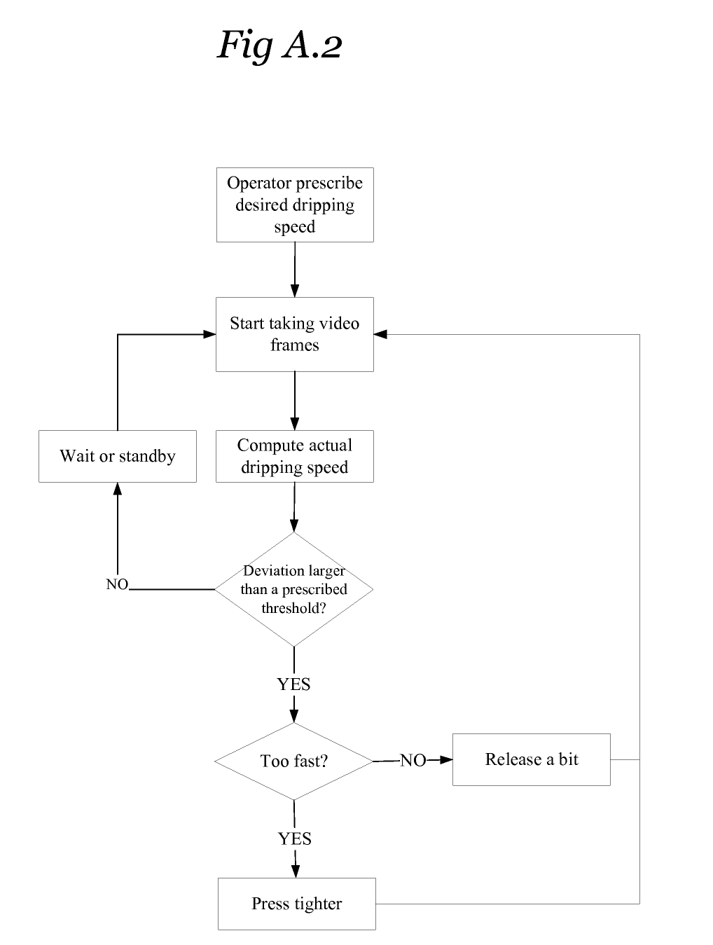

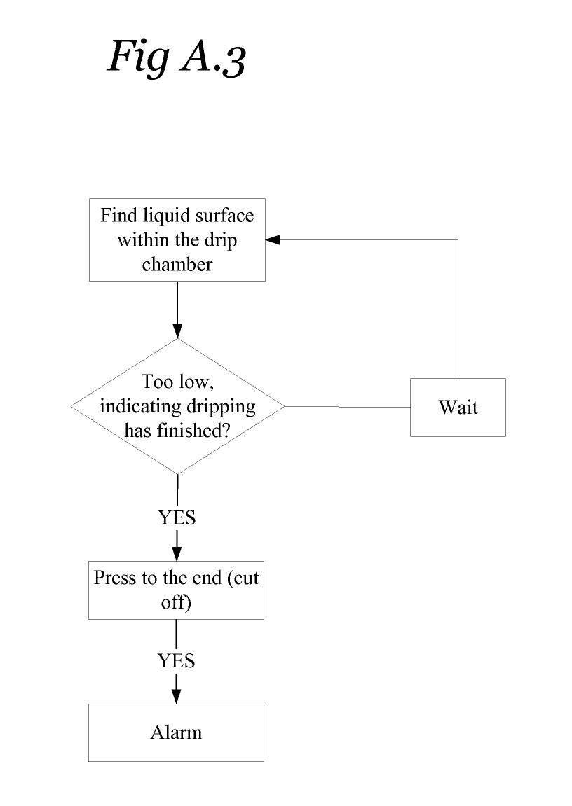

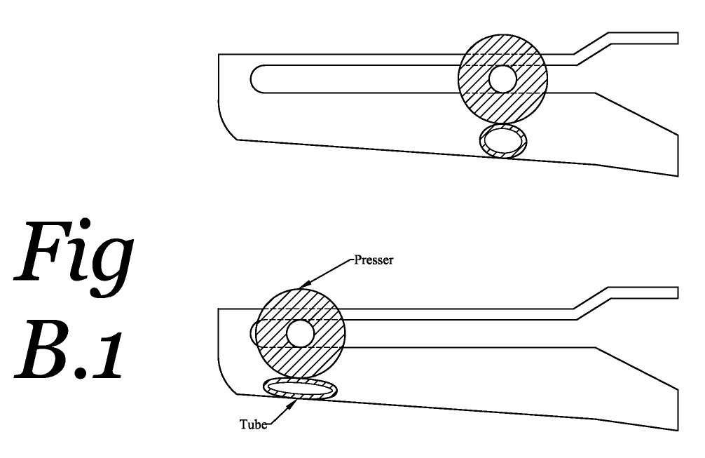

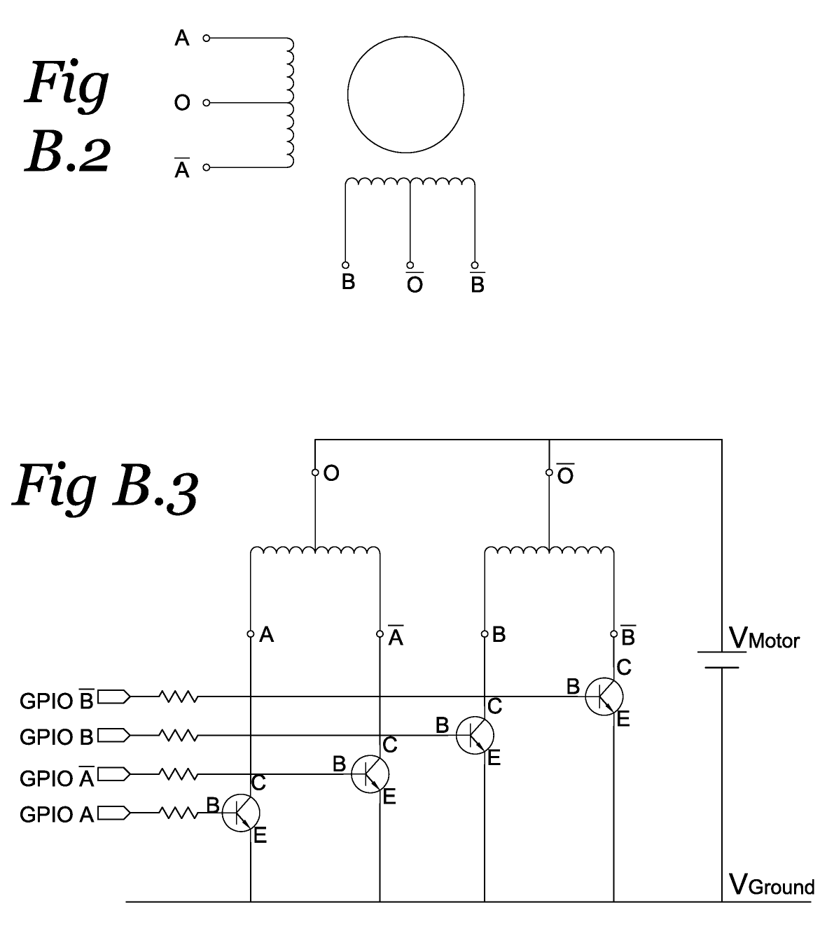

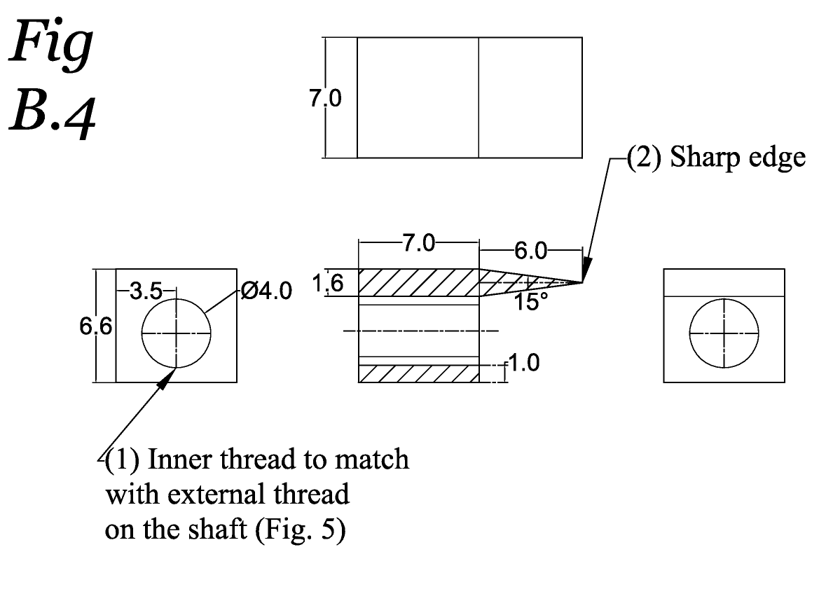

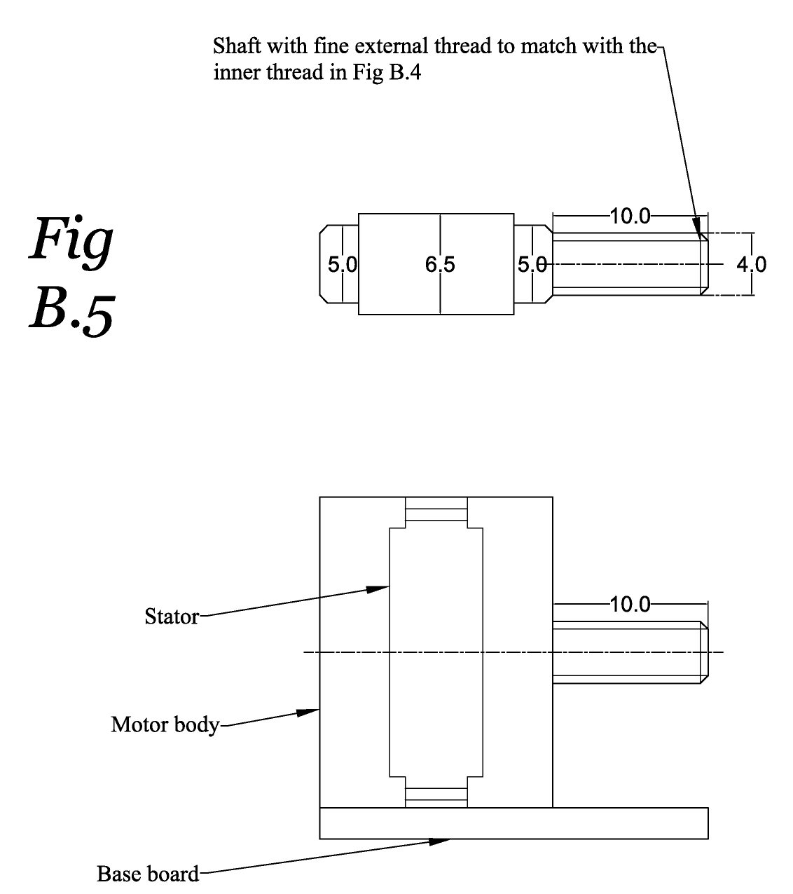

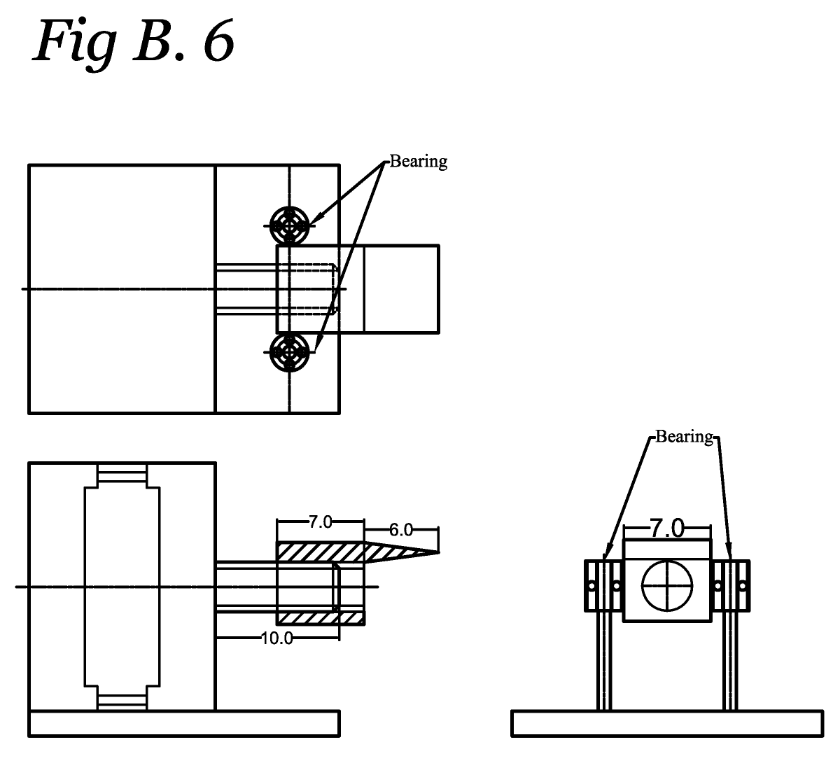

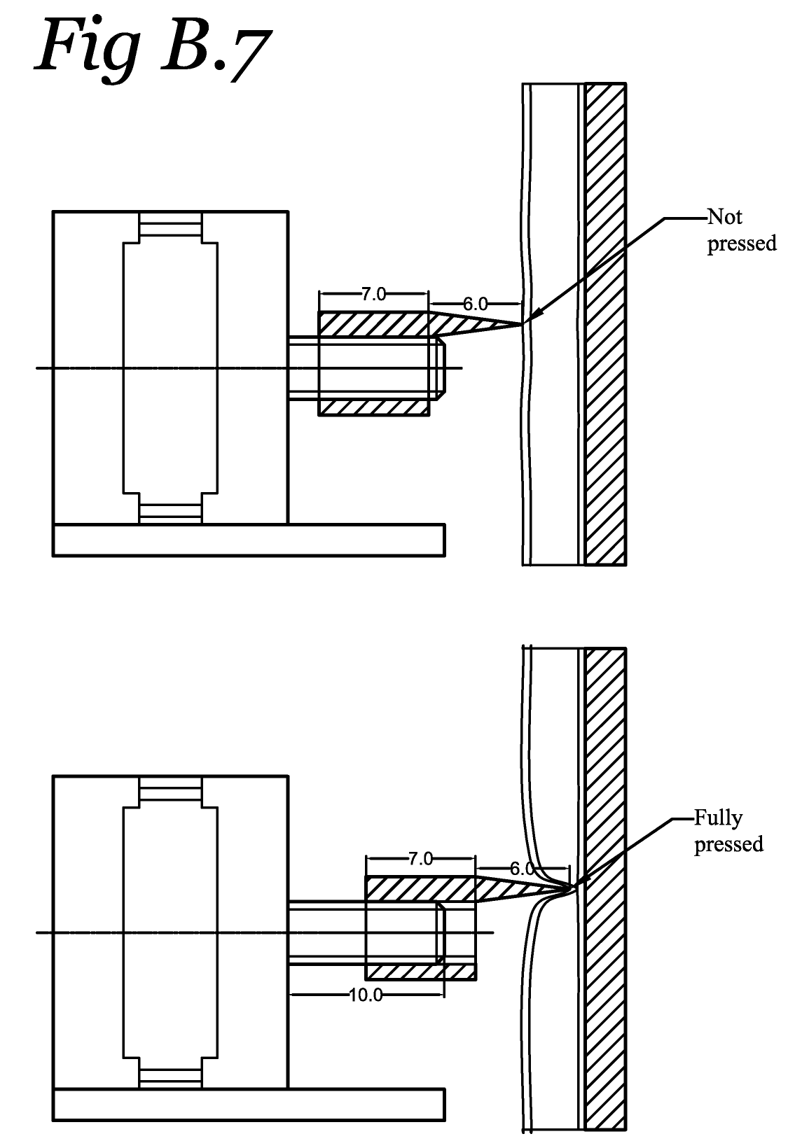

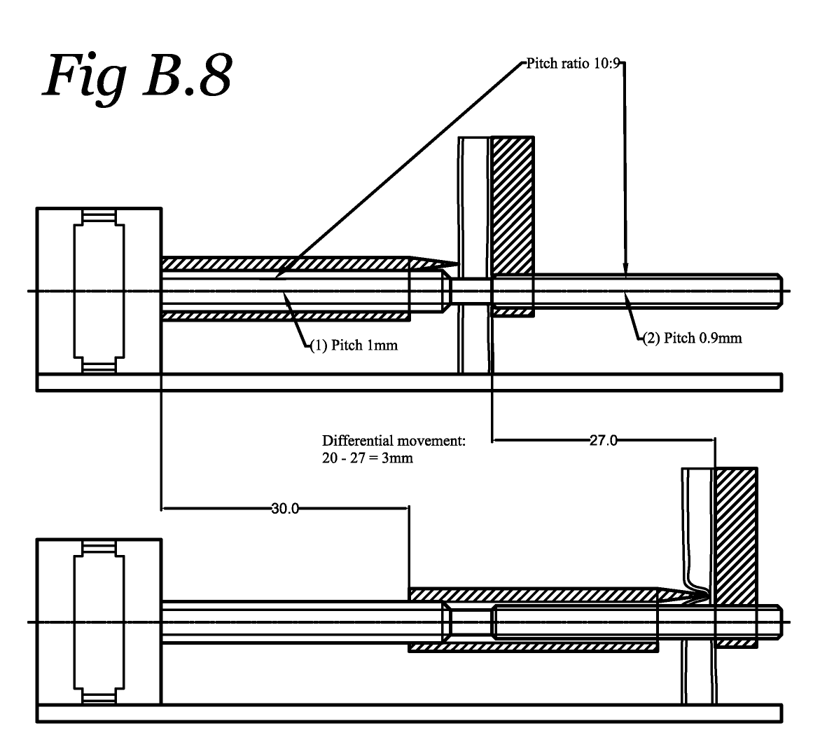

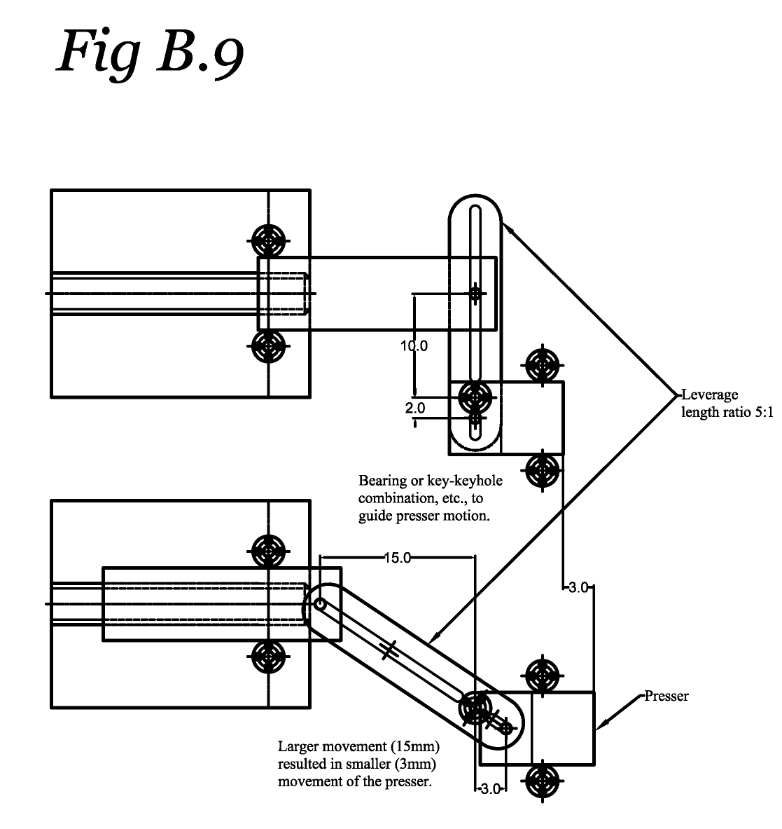

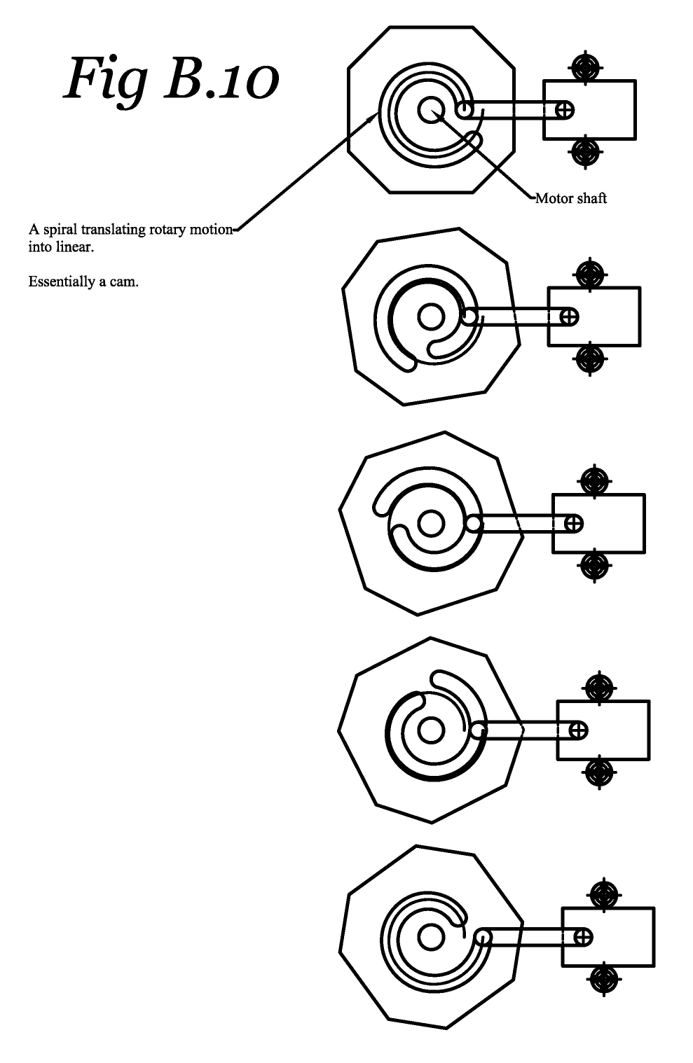

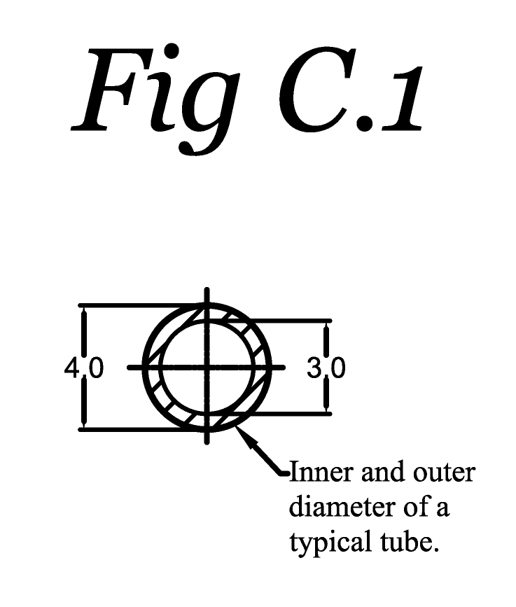

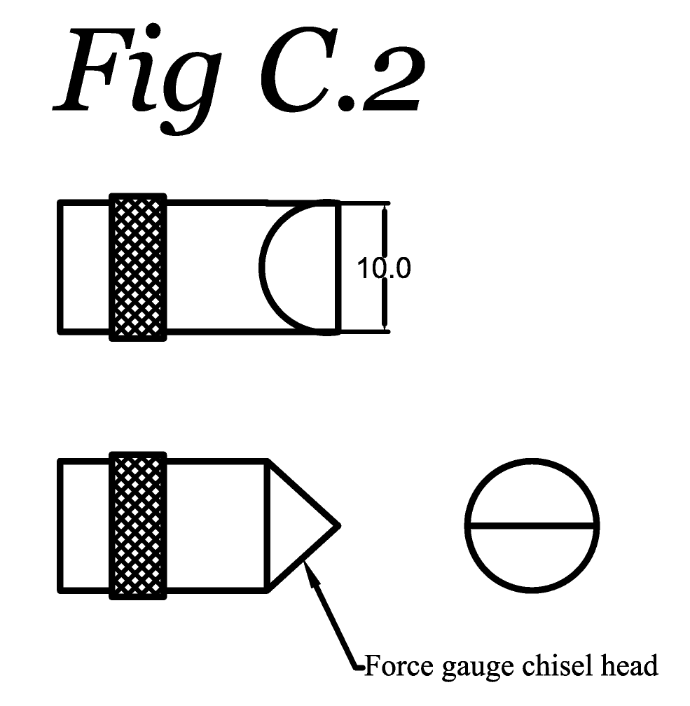

### CROSS-REFERENCE TO RELATED APPLICATIONS

Application No. 12825368: IV Monitoring by Digital Image Processing, by the same inventor

Application No. 12804163: IV Monitoring by Video and Image Processing, by the same inventor

### FEDERALLY SPONSORED RESEARCH

Not Applicable

### THE NAMES OF THE PARTIES TO A JOINT RESEARCH AGREEMENT

Not Applicable

### SEQUENCE LISTING OR PROGRAM

Not Applicable

### BACKGROUND

1. Field of The Invention

This invention relates to monitoring and controlling of intravenous dripping system.

### BACKGROUND

2. Pior Art

IV therapy is widely used in for drug administration across the world. Among machines used to control the IV process, the most widely used types are infusion pumps. A typical infusion pump uses a peristaltic pump to control the liquid flow, thus eliminating the need for gravity.

There are several disadvantages of infusion pumps:

1. Since these pumps work by pressing the dripping tube with a fixed pattern over a long period of time, they usually require specially-made tubes of high resilience. These tubes are often several times more expensive than ordinary tubes.

2. The pumps themselves are also expensive, usually cost over $1,000 for each.

3. They consume power and produces noises in operation.

4. Most of them are large and heavy.

There have also been attempts to invent IV control system without mechanical pumps and to base it on gravity dripping. In order to achieve this, the prerequisite is to be able to measure the dripping speed accurately. There have also been many inventions attempting to solve this problem, for example

1. U.S. Pat. No. 4,383,252 Intravenous Drip Feed Monitor, which uses combination of a diode and phototransistor to detect drips.

2. U.S. Pat. 6,736,801 Method and Apparatus for Monitoring Intravenous Drips, which uses infrared or other types of emitter and a sensor combined to count the drips.

There have also been inventions to combine monitoring and controlling together, for example U.S. Pat. 6,981,960 Closed-loop IV fluid flow control, uses a fluid sensor placed on the fluid path to measure dripping speed, and uses peristaltic pump or controlled valve to control the dripping speed.

### SUMMARY

In application 12825368 and 12804163, the inventor has shown how video and image processing technique can be used to accurately measure the speed of IV dripping. In this invention, we show how an electromechanical device can be used to control the speed of IV dripping based on the measured speed.

The basic idea is to use a presser to press the tube in the same way as a manual adjuster. When the tube is being pressed to the end, the drip will be cut off and when it is fully released, the liquid drips down freely. Between these two extremes, the thickness of the tube could effectively control the dripping speed.

To achieve this precise control we use several electromechanical embodiments. What is common is that the mechanical parts are all controlled by a stepper motor, and the stepper motor is in turn controlled by the processing unit with pin signals. A simple circuit for stepper motor control is shown in the FIG. B.3.

To translate stepper motor's rotary motion into linear motion to press the tube, the preferred embodiment is to use a micro-leadscrew. In the specification we show that such leadscrew mechanism has at least three advantages:

1. It allows very fine movement control over a short distance.

2. It is self-locking. The pressure that the tube exert on the presser would not result in rotary motion of the shaft, so we can safely cut the motor's current off when a desired speed is achieved. This saves power and allows the device to be driven by batteries.

3. The leadscrew can magnify force so that a small-sized stepper motor with relatively small torque can be used to drive the presser. This allows us to minimize the footprint of the device.

In addition to the basic leadscrew presser, we also show two of its variants:

1) A differential leadscrew combination.

2) Leadscrew-Lever combination.

These two variants provide higher precision to better accommodate the non-linear relationship between tube thickness and dripping speed.

A cam embodiment for translating stepper motor's rotary motion into linear motion is also shown in the invention. Since cams are not self-locking, we also describe methods for locking its position without the need for steady motor current.

### DRAWINGS—Figures

FIG. A.1 shows an embodiment of the invention. The upper part uses a camera and processing unit to measure the speed of dripping, and the lower part controls the dripping speed using a presser whose position is controlled by a stepper motor, which is in turn controlled by the processing unit. There is also an alarm built into the device for alarming the patient (attendant, nurse, etc.) when dripping has finished.

FIG. A.2 outlines a possible flowchart of the process. The measuring part and the controlling mechanism basically constitute a closed-loop control system in which the processing unit issues command to the motor based on the feedback from camera.

FIG. A.3 shows the flowchart for tube cut-off When the measuring part has detected that the dripping has finished, the processing unit would control the motor to push the presser to the end, thus prevent blood from flowing back into the tube. After that, it will somehow issue an alarm; if not, it would simply wait and detect later.

FIG. B.1 shows a common manual adjuster for IV control for reference and comparison.

FIG. B.2 shows the simplified circuit diagram of a stepper motor.

FIG. B.3 shows how controlling signals from the processing unit can be used to control the rotation of the stepper motor.

FIG. B.4 shows the drawing for a presser that will be mounted on the motor's threaded shaft to translate motor's rotation into linear motion to control the dripping speed.

FIG. B.5 contains two parts. The upper is a shaft with fine screw thread grinded on its front end. This external thread is to match with the internal thread in the presser of FIG. B.4. The lower part shows a view of the stepper motor when the threaded shaft has been assembled together with the other parts.

FIG. B.6 is the combination of FIG. B.4 and FIG. B.5. The presser's internal thread and the shaft's external thread are matched together. There are bearings on both sides of the presser to prevent it from rotating, thus permitting only linear motion.

FIG. B.7 shows how FIG. B.6 can be used to control the dripping. The upper part shows a position that the tube is not pressed and the lower part shows a position that the tube is fully pressed. For clarity of the view, the bearings for preventing rotation are omitted.

FIG. B.8 shows a variation of FIG. B.6 and FIG. B.7. Differential combination of two leadscrews are used and the effective pitch now is the difference of the pitches of the two leadscrews.

FIG. B.9 shows a variation of FIG. B.6 and FIG. B.7. A lever used here to achieve finer control of the linear motion and the pressing force of the leadscrew is magnified on the presser.

FIG. B.10 shows another embodiment of the controlling mechanism. A spiral groove is used to translate the motor's rotation into linear motion of the presser.

FIG. C.1 shows the sectional view of a common plastic tube. The inner diameter is 3 mm and the outer diameter is 4 mm These two values need to be considered in the leadscrew and presser design.

FIG. C.2 shows a force gauge's chisel head which has been used to gauge the maximum force needed during the pressing of the tube. This is important in determining the parameters of the motor as well as the material of the shaft and presser.

### DRAWINGS

Reference Numerals

In FIG. A.1

1 Containing box

1 Camera

1 Processing unit

1 Light source

5 Drip chamber

6 Tube

7 Stepper motor

8 l Presser

9 Alarm

### DETAILED DESCRIPTION

Introduction

In application 12825368 and 12804163 we have shown how video and image processing technique can be used to measure the speed of IV dripping. To summarize, we extract periodical information, such as the height of the drip, from a number of frames and compute the Discrete Fourier Transform of the vector. The index of the component with the largest magnitude (other than the constant component) gives the number of drips that has fallen during the interval. For validity and accuracy of this technique please refer to the said two applications.

Based on an accurate measurement of the speed, we invented an electromechanical device to control the speed of the dripping. There are different embodiments of the controlling mechanism, and their combination with the measuring technique essentially constitute a closed-loop control system. The user (nurse, etc.) can prescribe a desired speed of dripping, and the device will constantly measure the actual speed of the dripping, adjusting the position a presser that is pressing the dripping tube if there is any deviation. Of course, it could also stop the dripping after it has finished to prevent blood from flowing back into the tube.

The purpose, in general, is to provide device with sufficient accuracy and affordable price to be suitable for mass application, especially in developing countries the high price of infusion pumps has made them prohibitive.

The detailed description of the invention is shown below along with the figures.

Unit

All units in this application are metric.

FIG. A.1—One Possible Embodiment of the System

The upper part the figure shows the measuring part of the system and it has been described in detailed in patent application 12804163. A camera takes video frames of the dripping and send that information to the processing unit, by means such as interrupt or simply storing them in the memory. The processing unit uses the algorithm in application 12804163 to measure the dripping speed.

An alarm, by audio, light, wired or wireless signal or other means, can also be added to tell the patient (nurse, attendants, etc.) that the dripping has finished.

The lower part of this figure shows how a presser is being driven by a stepper motor to press the tube. The idea is straightforward and imitates the way of a manual adjuster shown in FIG. B.1. If the tube is released, gravity will result in the dripping; to make it slower or stop it, we can simply press the tube tighter or to the end.

FIG. A.2—Flowchart of Control

This figure outlines the basic procedure for dripping speed control. The user would first prescribe a desired speed value and the device will constantly measure the actual speed and compare it with the prescribed value. If the speed is too fast, it would control motor to press the tube tighter and would release the tube a bit if the speed is too slow. Once the deviation between the actual speed and the prescribed speed is with an acceptable threshold, it would keep the position of the presser.

That we are measuring the actual speed constantly is because it can be affected a number of factors. For example, if the patient moves the position of his hand (or other body parts that the liquid is infused into), or if the liquid bottle has been moved, or if the tube has been pressed by other objects (patient's body, etc.), or the drop of liquid height has resulted in a change in the liquid pressure, then in all these cases the speed could possibly get changed.

The problem of finding quickly the position of the presser corresponding to the desired speed should falls in general to the category of automatic control convergence, and there exist many algorithms for this, which are already the state of the art and we are not going to discuss it here.

To save power, the device could also choose to perform the task periodically, say, every 5 minutes rather than constantly, and turn into standby or some other power-saving mode during the interval. In conjunction with the property of leadscrew that linear force would not be converted into rotary (provided that the friction is large enough) for which the maintaining torque from the motor is not needed, this scheme allows us to build a very power-efficient device that could sustain for hours based only on batteries. We would discuss this in detail later.

FIG. A.3—Flowchart for Tube Cut-off

FIG. A.3 shows the flowchart for dripping cut off When the measuring part has detected that the dripping has finished, the processing unit would control the motor to push the presser to the end, thus prevent blood from flowing back into the tube; if not, it would simply wait and detect later. The technique for detecting the surface level is described in application 12825368 of the same inventor. Basically, it works by first converting the image into binary, then fining out the position of the liquid surface corresponding to the row with the highest number of white pixel count. For its implementation details please refer to the said application.

In the case that it has detected the finishing of the dripping, in addition to cutting off the tube, the device can also alarm patient (nurse, attendant, etc.) this event. Please refer to FIG. A.1 for description of the alarm.

FIG. B.1—A Manual Adjuster

This shows the profile of a manual adjuster which we have already mentioned in the description of FIG. A.1. For such a manual adjuster, the nurse (or patient, etc.) would roll the wheel to a position so that the tube is appropriately pressed to allow the drips to fall at a certain speed. The basically principle for embodiments of our controlling mechanisms are all similar to it.

FIG. B.2—Circuit Diagram of a Stepper Motor

This figure shows circuit diagram that are commonly seen in stepper motor manuals. There are two coils for electromagnets. Each coil has a middle end, denoted as O and Ō. By reversing current direction stepper motors can be controlled to rotate either in clockwise or anti-clockwise direction, and the two middle ends O and Ō allow the motor to be controlled by very simple circuits.

FIG. B.3—Circuit for Stepper Motor Control

This figure shows a simple circuit to control the stepper motor and this has been widely known to people working with them. The middle ends of the two coils are connected to a higher voltage, typically the nominal voltage of the motor, and for each coil the two other ends are connected to the ground through the collector and emitter ends of a transistor. Signals from the processing unit, for example, via GPIO (general-purpose input-output pins), are used to control the transistor. The resistors in the base ends are chosen so that once the transistors are turned on, they are working in saturation mode. The ways of how these controlling signals need to be synchronized are widely available in literatures.

One thing needs to bear in mind is that since, according to the diagram, when current flows through the coil only half of the coil is actually used. For example, when GPIO A is driven high by the processing unit, the current will flow through O-A-C-E, rather than the full coil ĀA . Therefore, if we allow only the nominal maximum current to pass through half of the coil, the maximum torque obtainable is only 1/2 of the maximum torque in the manual.

FIG. B.4—Presser

In this figure we show the shape and dimensions of a possible presser embodiment. There are four drawings here corresponding to top, back, side and front view of the presser. Although there can of course be variations on the presser's shape and construction, there are several important points to keep in mind:

1) In the side view we see that the angle of shaft edge is only 15° and we have added the annotation that it needs to be made sharp. Why? From the theoretical point of view, the sharper the edge is and the smaller the angle, the smaller contact area there will be, hence the larger pressure. Experimentally, we have also experimented with different shapes of the presser and indeed have found that sharper the edge is and the smaller the angle, the less torque from the motor it requires to drive the presser.

2) FIG. C.2 is the drawing of a chisel head of a force gauge. The angle is only slightly smaller than 90° and the edge is moderately sharp. Our experiment has shown that the peak force during the pressing of a PVC plastic tube, measured when this chisel head is installed on the force gauge, is approximately 15N. With larger angles or even flat surface, it becomes more and more difficult or impossible to stop the dripping, even if raising the force several times.

3) The width of the edge should be larger than the width of tube when it is fully pressed. At that point, the tube deforms due to the pressure and its width in the dimension parallel to the presser edge widen. If the width of the presser edge is not large enough, one or two ends of the tube in that dimension will be pressed out of the width of the presser edge and hence will have no force exerted on these parts. Due to material resilience, it will then partly recover to its original shape will allow drips to pass through. If this happens, it would be very difficult or impossible to control the dripping speed. Common plastic tubes usually have a inner diameter of 3 mm and outer diameter of 4 mm, and the 7 mm edge width in this figure is determined by:

a. Experiment. For tubes of the above measurement, we have never found any of them has its width widen to over 7 mm in the presser edge dimension.

b. Calculation. Half of the outer circumference is πD/2=π4/2=6.28 <7 mm, therefore 7mm is larger than the largest possible width.

4) We also see in this figure that the length of the presser edge in the shaft direction is 6mm There is no particular reason for picking 6 mm From FIG. C.1 we see that the inner and outer diameters for a typical tube are 3 mm and 4 mm, respectively. The 6 mm length here is merely conveniently chosen to make it easier to design and construct.

The way the presser is driven is through its internal thread, which is to be matched with the external thread on the shaft. In FIG. B.6 we will see that there are bearings on both sides of the presser to prevent rotation, so it will only move in linear direction.

FIG. B.5—Threaded Shaft and Motor

In the upper part of this figure we see a motor shaft with grinded find external thread and in the lower part the stepper motor with the threaded shaft assembled.

What type of thread should we choose? There are square, acme as well as other shapes of threads for leadscrew use, however since the diameter of our stepper motor shaft is very small (for example, 4 mm as shown in the figure), it would be practically difficult or too expensive to make (for example, grind) these types of threads. For this reason, V shape threads are recommended for practical embodiment.

Due to electromagnetic aspect of their design consideration, stepper motors typically uses stainless steel as the shaft material. In practice, most of the shafts we have seen are made from 304 stainless steel. If they are to be made by grinding, grinders and abrasive wheel need to be chosen to avoid damaging or deforming the geometric shape of the shaft, in particular, to ensure that after grinding the threaded shaft is still symmetric with its axis.

There is also no particular reason why the threaded length on the shaft is 1 mm. It is chosen just to match with the dimensions of the presser in FIG. B.4 and to allow sufficient liner movement distance to effectively control (press) the tube.

FIG. B.6—Leadscrew Presser

The presser and threaded motor shaft are put here in combination. There are bearings two sides of the presser to prevent it from rotating so that it could only move in the linear direction when the shaft rotates. In this way, the presser can be controlled to press the tube according to the rotation of the motor. When both the dripping speed measurement and movement control of the presser are precise, this device allows very accurate control of the dripping speed.

We choose this micro-leadscrew design due to its three advantages:

4. It allows very fine movement control over a short distance.

5. It is self-locking. The pressure that the tube exert on the presser would not result in rotary motion of the shaft, so we can safely cut the motor's current off when a desired speed is achieved. This saves power and allows the device to be driven by batteries.

6. The leadscrew can magnify force so that a small-sized stepper motor with relatively small torque can be used to drive the presser. This allows us to minimize the footprint of the device.

To appreciate these advantages we need some calculation.

The formula of leadscrew from engineering textbook and manuals is a very close practical approximation of strict result obtained by calculus, and it is accurate enough for our use.

We have

$$\begin{matrix}{T}_{raise} = \frac{{Fd}_{m}}{2} ( \frac{l + π μ sec α {d}_{m}}{π {d}_{m} - sec α l} ) = \frac{{Fd}_{m}}{2} tan ( φ + λ ) & ( 1 ) \\ {T}_{lower} = \frac{{Fd}_{m}}{2} ( \frac{π μ sec α {d}_{m} - l}{π {d}_{m} + sec α l} ) = \frac{{Fd}_{m}}{2} tan ( φ - λ ) & ( 2 )\end{matrix}$$

Where T=torque F=load on the screw dₘ=mean diameter μ=coefficient of friction between external and inner thread material α=thread angle l=lead (thread pitch) φ=friction angle λ=lead angle

Derivation of this formula can be found from [Shigley's Mechanical Engineering Design, ISBN 0390764876, page 403-408].

If we choose lead l (thread pitch) to be 0.5 mm, and according to FIG. B.4 and FIG. B.5, the mean diameter dₘ can be approximated by 4 mm, we found that the lead angle λ=tan⁻¹ (l/πdₘ)=tan⁻¹(0.5/π4)=tan⁻¹(1/8π) ≈tan⁻¹(0.04) which is a very small angle. This is in fact the mathematical condition that the effective friction coefficient can be approximated by μ sec α, and we see here that our choice of l and dₘ do satisfy this condition.

Expanding tangent function in (1) and (2) by trigonometric equation, we have

$$\begin{matrix}tan ( φ + λ ) = \frac{tan φ + tan λ}{1 - tan φtan λ} & ( 3 ) \\ tan ( φ - λ ) = \frac{tan φ - tan λ}{1 + tan φtanλ} \\ tan φ = μ sec α & ( 4 )\end{matrix}$$

If we choose M-shaped (ISO metric standard) screw thread such that the V cut is 60°, then α=30° and sec α=1.1547 , and for metal materials usually μ is usually between 0.1 and 0.3. In the denominator of (3) and (4), tan φ tan λ≦0.3×1.1547×0.04≦0.014, so that 1±tan φ tan λ can be safely approximated by 1.

What is the utility of these equations? Replacing (3) and (4) into (1) and (2), we have

$$\begin{matrix}{T}_{raise} = \frac{{Fd}_{m}}{2} ( \frac{l + π μ sec α {d}_{m}}{π {d}_{m} - sec α l} ) = \frac{{Fd}_{m}}{2} \frac{tan φ + tan λ}{1 - tan φtan λ} & ( {1}^{′} ) \\ {T}_{lower} = \frac{{Fd}_{m}}{2} ( \frac{π μ sec α {d}_{m} - 1}{π {d}_{m} + sec α l} ) = \frac{{Fd}_{m}}{2} \frac{tan φ - tan λ}{1 + tan φtanλ} & ( {2}^{′} )\end{matrix}$$

From (1), we can compute the maximum torque of the motor needed to drive the presser. For our choice of values:

$$\begin{matrix}{T}_{raise} = \frac{{Fd}_{m}}{2} \frac{tan φ + tan λ}{1 - tan φtan λ} \\ \approx F \frac{4}{2} \cdot ( tan φ + tan λ ) \\ \approx F \cdot 2 \cdot ( tan φ + 0.04 )\end{matrix} \\ if \\ \begin{matrix}tan φ = 0.2 \\ = F \cdot 2 \cdot ( 0.2 + 0.04 ) \\ \approx F \cdot 0.48 \\ = F \cdot 0.5 ( N \cdot mm )\end{matrix} \qquad ( 5 )$$

Recall that in the discussion of FIG. B.4, we cited the experiment result using force gauge with chisel head in FIG. C.2, that the peak force during the pressing of the tube is 15N. Substituting this into (5), we have

$$\begin{matrix}{T}_{raise} = 15 \cdot 0.5 \\ = 7.5 N \cdot mm \\ = 0.75 N \cdot cm \\ \approx 75 gf \cdot cm\end{matrix} \qquad ( 6 )$$

This is in fact a very good result. Why? For anyone with the aim to build a practical device, the availability of components and their prices must be taken into consideration. In the discussion for FIG. B.3, we have shown that since only half of the coil of the stepper motor's electromagnet is used, its maximum torque will be reduced by half The torque in (6) we have calculated is exactly produced by these half coils, so the full maximum torque for the motor should be multiplied by 2, so we have

Tₘₒₜₒᵣ75 gf·cm×2=150 gf·cm (7)

This closely matches the maximum torque of standard 20 mm (both height and width) stepper motors easily found from suppliers. Steppers of this size usually have their torques ranging between 150 gf·cm and 400 gf·cm, and they are very cheap especially from manufacturers in China. There is basically no need for us to make customized motors to suit our needs. Their small size (20 mm) also allow for us to build device with very small footprint.

Also note that the above calculation was based on the 15N peak force which is measured using a chisel head with nearly 90° angle (see FIG. C.2) and moderately sharp edge. For smaller angle and sharper edge (see our 15° presser edge example in FIG. B.4), it is reasonable to expect that smaller force would be enough, thus further reducing the need on motor torque.

Among the three advantages of leadscrew embodiment we have mentioned, our calculation so far have just proved point 3. Formula (2) would allow us to under the second point:

$${T}_{lower} = \frac{{Fd}_{m}}{2} \frac{tan φ - tan λ}{1 + tan φtan λ} \qquad ( {2}^{′} )$$

As long as tan φ tan λ>0, there is always a torque needed to rotate the shaft in the reverse direction. Therefore, pressure in the shaft direction along is impossible to cause the revese rotary motion. For tan φ −tan >0 to be valid, we need

tan φ=μ sec α=μ·1.1547>tan λ≈0.04

∴μ>0.04/1.1547=0.0346 (8)

Practically, this is nearly always satisfied. It is in fact very difficult to find metallic material combination with friction coefficient close to this value.

Because of this, the leadscrew is self-locking. We can exploit this to save power by cutting off motor current when the prescribed dripping speed has been achieved. We the motor is in use, the current can be as large as 300 mA or more which could consume battery power quickly; but if we only let current pass through it at the instants when dripping speed adjustments are needed, then the majority of power can be saved as comparing to maintaining the position by a steady current.

In conjunction with this, the processor and camera can also be turned off or into power-saving mode when not in need and wake them up

1. Periodically

2. By sensor, such as when a movement is sensed so that the speed might have changed

There hence can be a variety of schemes of to save power efficiently, but not without the self-locking property of leadscrews.

Note that there are also bearings on the two sides of the presser and their friction, which is usually very small due to rolling friction, has been omitted in the above calculation. Ball, needle, or basic contact bearing can also be used if friction is acceptably small. If the motor has enough remaining torque, key and keyway combination or alike can also be used. Regarding the number of bearings, since geometrically it is evident that a single bearing in close contact with the presser could also effectively prevent the presser from rotating, a single bearing can also be used instead of two.

Next we show that the control is indeed very precise. Hybrid stepper motors typically have step angle of 1.8°. In our previous calculation the lead (thread pitch) was chosen to be 0.5 mm, and with each step of the motor the presser proceeds/reverses

$$0.5 mm \times \frac{1.8 °}{360 °} = 500 μ m \times \frac{1}{200} = 2.5 μ m \qquad ( 9 )$$

This would effectively divide the 3mm diameter of a common tube into

$$\frac{3 mm}{2.5 μ m} = \frac{3000 μ m}{2.5 μ m} = 1200 steps$$

far exceeding the precision of human's manual adjustment (see the manual adjuster in FIG. B.1).

Our experiment, however, show that the relationship between dripping speed and the tube's thickness (in the dimension of being pressed) is strongly non-linear. This differs with the shape, angle and edge sharpness of the presser, the material of the tube, and even its positioning with respect to the presser edge (orthogonal or not, etc.). In some cases, the perceivable change of dripping speed only happens when the thickness of the tube is being controlled between 0 5 mm and 0 (stopped). Even with this rather extreme situation, for this 0 5 mm thickness, we still have

$$\frac{0.5 mm}{2.5 μ m} = \frac{500 μ m}{2.5 μ m} = 200 steps$$

which is also precise enough for all practical considerations.

There are three types of stepper motors, namely 1) Permanent magnet type 2) Variable reluctance type 3) Hybrid type. Among them, hybrid type usually allows the smallest step angle and consequently the highest precision. The above calculation was based on a hybrid stepper motor, but did not rule out the possibility of other two types of stepper motors. In practical embodiment, as long as their precision meets the requirement, the other two types of stepper motors can also be used, especially

1) When differential leadscrew as in FIG. B.8 are used to provide higher movement precision, therefore reducing the precision requirement on stepper motors.

2) When leadscrew-lever combination as in FIG. B.9 are used to provide higher movement precision, therefore reducing the precision requirement on stepper motors.

To summarize, in this description of FIG. B.6, we have shown chiefly three advantages of the leadscrew embodiment from both theoretical and practical perspectives. We recommend this as a preferable embodiment of the invention.

FIG. B.7—Example

This figure shows an example when of a leadscrew presser is at work. In the upper figure the tube is not pressed and in the lower part it is fully pressed.

FIG. B.8—Differential Leadscrew

There can be variations based on the leadscrew presser of FIG. B.6. We have mentioned in the description of FIG. B.6 that the speed-thickness relationship is strongly non-linear and perceivable change could only happen when the thickness is between zero and value much smaller than the tube's inner diameter. To provide even finer control over this small range and also the full range, differential combination of two leadscrews are used. The shaft is divided into two parts and has thread of different lead (pitch) grinded on them. In this example, the ratio between the two pitch is 10:9. To achieve this, we can make the pitch on the presser and its corresponding shaft part to be 1.0 mm and on the tube fixture and its corresponding shaft part to be 0.9 mm. When these two threads are of the same handedness, the effective pitch for this configuration hence is 1.0−0.9=0.1 mm. To make them leadscrews, there of course needs to be bearings or key and keyway combination to prevent them from rotating, which are omitted in the image for visual clarity. In this figure's example, to fully press the tube to its diameter of 3 mm, the presser needs to be driven 30 mm to the right whereas the fixture with the tube are driven 27 mm to the right, and their relative movement results in the pressing of the tube.

Combinations like this are common in micro-mechanisms. In addition to the embodiment in the figure, one can also fix the position of the presser and let motor and tube-fixture to have relative movement, as well as other similar implementations.

FIG. B.9—Leadscrew-Lever Combination

This figure shows another variation based on the leadscrew in FIG. B.6 that could be used to enhance the precision. The additional component introduced is essentially a lever. In this embodiment, the presser is not an integral part of the nut as in previous embodiments. The nut of the leadscrew has a small cylindrical connector that is fitted into the groove in the lever and the presser also has a same connector fitted into the groove. Bearing, key-keyway combination or other mechanism is used to guide and restrict the motion of the presser to on the line parallel to, but in the reverse direction of the motion of the nut. The ratio between the two arms to the pivot is always 5:1 in the example, and by the principle of lever:

1. One unit of linear movement of the presser would require 5 units of movement of the nut, thus enhancing the precision by 5 times.

2. The linear driving force of the nut is magnified by 5 times on the presser, thus allowing motors with even smaller torque to be used.

As in previous embodiments, rolling friction between nut/presser and the bearing are not calculated, as well as for the friction between the cylindrical connector and lever groove. The connector, of course, can also be built on bearing, further reducing the friction.

The lever length ratio shown here are only for illustrational purposes. Levers can be classified into three classes according to the relative position of the fulcrum, the load and the force and the type that the load is between the force and the fulcrum can also be used.

FIG. B.810—Cam Embodiment

The last embodiment we show is a cam embodiment. There are numerous types of cams and we have shown in this embodiment a spiral. There are five positions shown here to show how rotation of the cam would drive the linear motion of the presser.

In each of the small figures, the central circle in the front view is the motor shaft. A board is connected to the shaft and a groove is cut on the board. The geometric shape of the groove is the envelope a circle running with its center moved along a spiral curve. If the groove rotates in the clockwise direction, the presser will be pressed to the right through the cylindrical connector that is fitted into the groove, and will be pulled to the left if the groove rotates in the anti-clockwise direction.

If indeed as in the present embodiment that an Archimedean spiral is used, since spiral's polar equation is:

r=C+αθ (10)

C is always a constant. If we want one full rotation to cause the presser to move a distance of 3 mm, which is the inner diameter of a typical tube, then

$$3 mm = Δ r = αΔθ = α \cdot 2 π \\ ∴ α = \frac{3 mm}{2 π} \qquad ( 11 )$$

We can in this way calculate parameters of the spiral. For ease of manufacturing such that the groove's two ends would not touch each other, we might also prefer to choose rotation smaller than a full circle.

The relationship between rotary and linear motion translation from a spiral cam is linear. To better accommodate the non-linear relationship between tube-thickness and dripping speed, we could also design cams of other shapes based on experiments and calculation.

There is an important difference between cam embodiment and leadscrew embodiment (and its differential and lever variations):

1. Leadscrew embodiment and all its variations are self-locking.

2. Cam embodiments are not self-locking. Steady current needs to be maintained, or other types of brake might be used to lock its position without further supplying energy.

To lock the cam without continuous current, one might, in the following steps:

1) Use gear combination to magnify the rotation such that 1.8° rotation would result in a much larger rotation of a plate. The plate has slots or holes evenly spanned in different directions.

2) Use an electromagnet to lift a small object connected to a spring. When the electromagnet is off, the spring will push the small object into a hole or slot of on the plate, therefore locking its position; when the electromagnet is on, it will lift the small object up and the plate, consequently the motor shaft and cam, would be allowed to move again.

Using the same electromagnet with rubber-spring combination is also possible. Rather than holes on the plate, friction of the rubber could also prevent the cam and shaft from rotating.

FIG. C.1—Sectional View of a Tube

In this figure we show the inner and outer diameter of a typical tube. These values have been used in various places of calculation in our specification.

FIG. C.2—Chisel Head of a Force Gauge

In this figure we show the chisel head of a force gauge which we have used to measure the peak force during pressing of the tube.

Statement

We make it clear that numerical values, particularly regarding the dimensions of mechanical components in this disclosure, are provided for clarity of illustration and to help people skilled in the field to make and use this invention, rather than to impose any limitation on the scope of the invention. The scope of this invention is only specified in the claims.

What is claimed is:

1. An electromechanical device for IV control, comprising
a) A camera and a processing unit.
b) A stepper motor, either of hybrid, permanent magnet or variable reluctance type.
c) A threaded motor shaft.
d) A presser with internal thread matching the external thread on the motor shaft.
e) Mechanism to prevent the presser from rotating.
f) A fixture to hold the tube.
g) An alarm to signal events.
And a process for controlling the dripping speed, which works by
1) Measure actual dripping speed.
2) Comparing actual speed with prescribed speed
3) Press the tube with the aforementioned presser tighter or release a bit accordingly.
4) Repeat if the deviation is larger than threshold.
And a method for cutting off the tube, which works by
1) Detecting whether dripping has finished.
2) Press the tube to the end with the aforementioned presser if true, or wait if otherwise.
3) Alarm the patient (nurse, attendant) of this event.

2. An electromechanical device of claims 1, with the claim 1's shaft and nut combination replaced by differential combination of leadscrews.

3. An electromechanical device of claims 2, with its differential leadscrew implemented by
a) A stepper motor whose position is fixed.
b) A leadscrew to change the position of the presser.
c) A leadscrew with different thread pitch to change the position of the tube fixture.

4. An electromechanical device of claims 2, with its differential leadscrew implemented by
a) A presser whose position is fixed
b) A leadscrew to change the position of the motor.
c) A leadscrew with different thread to change the relative position of the tube-fixture to the motor.

5. An electromechanical device of claims 2, with its differential leadscrew implemented by
a) A tube-fixture whose position is fixed
b) A leadscrew to change the position of the motor.
c) A leadscrew with different thread to change the relative position of the presser to the motor.

6. An electromechanical device of claims 1, adding a lever between claim 1's nut and the presser to enhance precision and to magnify force.

7. An electromechanical device of claims 6, in which the fulcrum is located between the leadscrew nut and the presser.

8. An electromechanical device of claims 6, in which the presser is located between the leadscrew nut and the fulcrum.

9. An electromechanical device for IV control, comprising
a) A camera and a processing unit.
b) A stepper motor, either of hybrid, permanent magnet or variable reluctance type.
c) A cam whose rotation is controlled by the stepper motor
d) A presser attached as the follower to the cam.
e) An alarm to signal events.
And a process for controlling the dripping speed, which works by 1) Measure actual dripping speed.
2) Comparing actual speed with prescribed speed
3) Press the tube with the aforementioned presser tighter or release a bit accordingly.
4) Repeat if the deviation is larger than threshold.
And a method for cutting of the tube, which works by
1) Detecting whether dripping has finished.
2) Press the tube to the end with the aforementioned presser if true, or wait if otherwise.
3) Alarm patient (nurse, attendant) of this event.

10. An electromechanical device of claims 9, in which the cam takes a spiral shape to result in linear relationship between motor's rotation and follower (presser)'s movement.

11. An electromechanical device of claims 9, in which the cam takes shape(s) to specifically accommodate the non-linear relationship between tube thickness and the dripping speed.

12. An electromechanical device of claims 9, in which the cam's position is allowed to be fixed without a steady motor current.

13. An electromechanical device of claims 12, in which the cam's position is fixed by
a) A brake of rubber or other material of high friction.
b) A spring to exert pressure on the brake so that the brake would prevent the cam or its associated components (shaft, etc.) from rotating.
c) An electromagnet to lift the brake to allow the cam to rotate.

14. An electromechanical device of claims 12, in which the cam's position is fixed by
a) A plate with evenly spanned holes or slots whose rotation is associated with that of the cam.
b) A spring to press small objects with suitable size into the holes or slots of the said plate to prevent the cam from rotating.
c) An electromagnet to lift the small objects to allow the cam to rotate.

15. An electromechanical device of claims 14, in which there are mechanism to magnify the rotation so that stepper motor's small rotation results in a magnified rotation of the plate, allowing smaller rotations to be fixed and easier manufacturing of the plate.
